(19) United States
(12) Patent Application Publication
(10) Pub. No.: US 2025/0069735 A1
(43) Pub. Date: Feb. 27, 2025

(54) COMPENSATING THE IMPACT OF POSITIONING ERRORS ON THE CERTAINTY AND SEVERITY GRADE OF FINDINGS IN X-RAY IMAGES

(71) Applicant: KONINKLIJKE PHILIPS N.V., EINDHOVEN (NL)

(72) Inventors: Jens von Berg, HAMBURG (DE); Sven Kroenke-Hille, HAMBURG (DE); Stewart Matthew Young, HAMBURG (DE); Daniel Bystrov, HAMBURG (DE); Heiner Matthias Brueck, PINNEBERG (DE); André Gooßen, ELDENA (DE)

(21) Appl. No.: 18/720,693

(22) PCT Filed: Dec. 16, 2022
(86) PCT No.: PCT/EP2022/086421

§ 371 Date: Jun. 17, 2024

Related U.S. Application Data
(60) Provisional application No. 63/292,609, filed on Dec. 22, 2021.

(51) Int. Cl. G16H 40/63; G06T 7/00; G16H 30/40

(52) U.S. Cl. CPC G16H 40/63; G06T 7/0012; G16H 30/40; G06T 2207/10116; G06T 2207/30008

(57) ABSTRACT

A computer-implemented method of compensating for image quality issue in an image study. The method includes acquiring a current image study, measuring, via a processor, a patient positioning parameter indicating a deviation of a patient position in the current image study to an optimal patient position for an image exam type of the current image study and analyzing, via an analysis module, the current image study and the determined patient positioning parameter to determine a relationship between positioning errors and a grading of a diagnostic finding for the current image study, wherein the grading of the diagnostic finding includes one of a severity of the diagnostic finding or a certainty classification of the diagnostic finding.

### BACKGROUND

Radiological examinations including medical image studies such as, for example, X-rays, are often the most efficient method for diagnosing and/or treating certain conditions. In some cases, however, deviations in patient positioning from an optimal positioning for a given X-ray exam type may affect a quality of the image exam and impact the diagnosis of the radiograph. For example, deviations from the ideal positioning may alter an appearance or measurable properties of an image that lead to a finding indication and diagnosis statement, mimic such a finding, cover such a finding, influence an apparent severity of a finding, or limit a certainty that a finding is reported with. Only very experienced radiologists may be able to cancel out this effect and assign a consistent and appropriate certainty estimate to their finding. For less experienced radiologists, deviations in image quality may result in errors in diagnosis.

### SUMMARY

Some exemplary embodiments relate to a computer-implemented method of compensating for image quality issue in an image study. The method includes acquiring a current image study, measuring, via a processor, a patient positioning parameter indicating a deviation of a patient position in the current image study to an optimal patient position for an image exam type of the current image study and analyzing, via an analysis module, the current image study and the determined patient positioning parameter to determine a relationship between positioning errors and a grading of a diagnostic finding for the current image study, wherein the grading of the diagnostic finding includes one of a severity of the diagnostic finding or a certainty classification of the diagnostic finding.

Other exemplary embodiments relate to a system compensating for image quality issue(s) in an image study. The system includes a non-transitory computer readable storage medium storing an executable program and a processor executing the executable program which causes the processor to acquire a current image study, measure a patient positioning parameter indicating a deviation of a patient position in the current image study to an optimal patient position for an image exam type of the current image study and analyze the current image study and the determined patient positioning parameter to determine a relationship between positioning errors and a grading of a diagnostic finding for the current image study, wherein the grading of the diagnostic finding includes one of a severity of the diagnostic finding and a certainty classification of the diagnostic finding.

Still further exemplary embodiments relate to a non-transitory computer-readable storage medium including a set of instructions executable by a processor. The set of instructions, when executed by the processor, causing the processor to perform operations including acquiring a current image study, measuring, via a processor, a patient positioning parameter indicating a deviation of a patient position in the current image study to an optimal patient position for an image exam type of the current image study and analyzing, via an analysis module, the current image study and the determined patient positioning parameter to determine a relationship between positioning errors and a grading of a diagnostic finding for the current image study.

### BRIEF DESCRIPTION OF THE DRAWINGS

FIG. 1 shows a schematic diagram of a system according to an exemplary embodiment.

FIG. 2 shows a flowchart of a method for compensating for deviations in a patient position from an optimal patient position for a particular image exam type according to an exemplary embodiment.

FIG. 3 shows a flowchart of a method for compensating for deviations in a patient position from an optimal patient position for a particular image exam type according to another exemplary embodiment.

FIG. 4 shows a flowchart of a method for compensating for deviations in a patient position from an optimal patient position for a particular image exam type according to yet another exemplary embodiment.

FIG. 5 shows examples of X-ray images including apparent findings influenced by a positioning of the patient.

### DETAILED DESCRIPTION

The exemplary embodiments may be further understood with reference to the following description and the appended drawings, wherein like elements are referred to with the same reference numerals. The exemplary embodiments relate to systems and methods for compensating for image exam quality issues resulting from deviations in a patient positioning from an optimal positioning for a given image (e.g., X-ray) exam type (e.g., chest, musculoskeletal). In particular, exemplary embodiments describe a system and method for determining a certainty classification and/or a grading for a finding, when the patient positioning deviates from the optimal positioning for the given X-ray exam type. Grading may be a number (e.g., heart size, lung-heart ratio) or a verbal statement (e.g., no, mild, moderate, prominent, strong) indicating the severity of a finding. Certainty classifications may include attributes of a finding such as, for example, suspected, possible, probable, almost certain, and certain. It will be understood by those of skill in the art that although the exemplary embodiments show and describe X-ray image exams, the system and method of the present disclosure may be applied for image exam types of any of a variety of modalities.

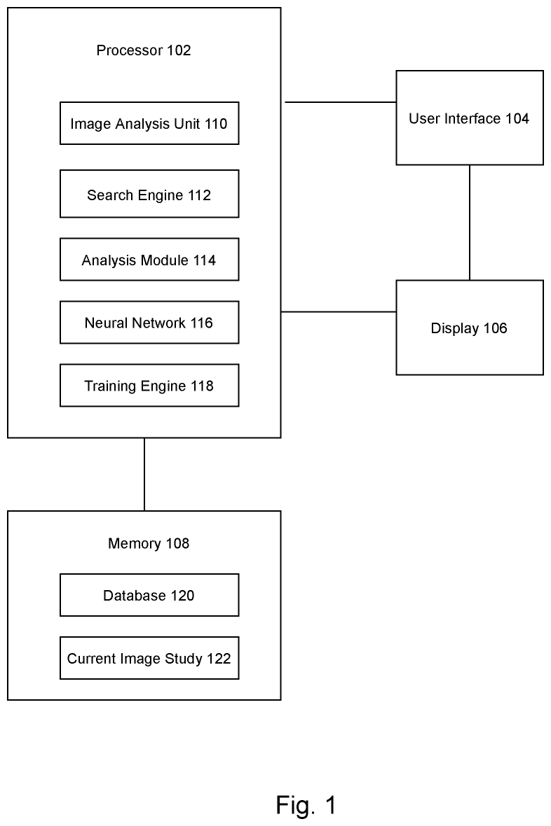

As shown in FIG. 1, a system 100 according to an exemplary embodiment compensates for deviations in patient positioning from an optimal positioning for a given exam type by retrieving previous exam studies with similar positioning parameters. The retrieved previous exam studies may be used to make a determination regarding a diagnostic certainty and/or a grading of a finding for a current image study. In a further embodiment, these determinations regarding diagnostic certainty and/or grading may be used to subsequently train a neural network 116 to automatically determine a certainty classification and/or grading for a given exam study to compensate for deviations in patient positioning. The system 100 comprises a processor 102, a user interface 104, a display 106 and a memory 108. The processor 102 includes an image analysis unit 110 for estimating patient positioning parameters for a given exam study, a search engine 112 for retrieving previous exam studies and an analysis module 114 for determining the certainty classification/grading of a finding. In a further embodiment, the system 100 includes the neural network 116 and a training engine 118 for training the neural network 116.

Throughout this description, the term “neural network” is used to describe a system comprising a plurality of processing nodes that are densely interconnected. Typically, the neural network is organized into layers of nodes but it is not a requirement. In the layer model, for example, a node may be connected to one or more nodes in a lower layer, from which it receives data, and one or more nodes in a higher layer, to which it sends data. It should also be understood that a neural network is a subset of machine learning, which is a method of data analysis that automates analytical model building. Thus, throughout this description, it should be understood that use of a neural network is only exemplary and any functions or operations described as being performed by a neural network may be performed by any type of machine learning and should not be interpreted as being limited to only a neural network.

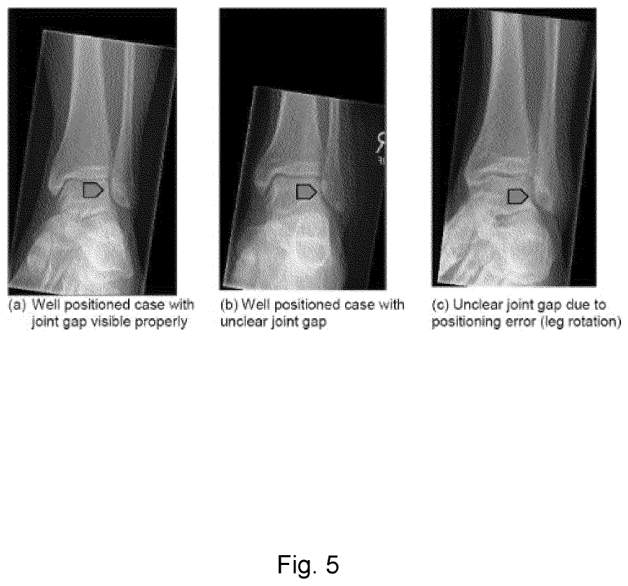

The image analysis unit 110 is configured to estimate patient positioning parameters (or a relevant subset thereof) for a given image exam type. Image exam types may include, for example, an X-ray of the chest or an X-ray of a bone. Patient positioning parameters for an X-ray of the chest may include, for example, a rotation around a patient's long axis and an inspiration status of the patient when the image was acquired. For example, the heart shadow of a patient may appear larger or smaller on a chest X-ray if the patient is rotated. In another example, lung opacities may appear much more prominent or may start to appear if the patient did not breathe in properly as the parenchyma is denser. The image analysis unit 110 may automatically measure deviations in rotation and/or inspiration from an ideal position. Patient positioning parameters for bone X-rays may include, for example, geometrical properties such as differing angles and distances in a three-dimensional space with respect to intrinsic coordinates of, for example, a joint as well as its flexion. For example, a joint gap in a musculoskeletal imaging may appear pathologically too narrow if the angle of incidence from the X-ray is not going through it as required when following the guidelines, as shown in FIG. 5. FIG. 5 shows exemplary findings of a narrowing of the fibula-talus joint gap according to X-ray images of an ankle joint, which may, for example, be used to train the neural network. For example, image (a) shows a well-positioned ankle joint in which a joint gap is properly visible, image (b) shows a well-positioned angle joint in which the joint gap is unclear due to disease and image (c) shows an unclear joint gap resulting from a sub-optimal positioning of the leg and/or foot. In another example, subtle bone fractures may be partially covered or hardly visible if the angle of X-ray incidence on the fracture surface is close to 90 degrees or if other bone overlay the fracture in the projection plane. The image analysis unit 110 may automatically measure and/or estimate deviations in pose and posture of a joint based on the geometrical properties.

The search engine 112 may operate on, for example, a Picture Archiving and Communication System (PACS) of the imaging device (e.g., X-ray device). In one embodiment, the search engine 112 may retrieve previous exam studies from a database 120, which may be stored to the memory 108 of, for example, the imaging device. These retrieved previous exam studies may be displayed to the user on the display 106 of the system 100 or, alternatively, on a display of a computing system in network communication with the system 100. The search engine 112 is configured to retrieve previous imaging exam studies of the same type as a current study 122 being read and having similarly positioned patients based on the patient positioning parameters identified via the image analysis unit 110. The search engine 112 may also be configured to apply additional search filters such as, for example, specific findings/diseases and/or specific patients.

The analysis module 114 may include a graphical user interface accessible to the user (e.g., radiologist) via the user interface 104 for a manual and/or (semi-) automatic compensation for deviations in patient positioning from an optimal positioning. The user interface 104 may include any of a variety of input devices such as, for example, a mouse, a keyboard and/or a touch screen via the display 106. In particular, the analysis module 114 permits the user to make manual determinations (inputs) with respect to a certainty classification (e.g., suspected, possible, probable, almost certain, and certain) and/or a severity grade (e.g., none, mild, moderate, prominent, strong) indicating the severity of a finding based on the previous exam studies retrieved via the search engine 112. Upon training of the neural network 116, however, the analysis module 114 may provide automatic and/or semi-automatic estimations with respect to certainty and/or severity of a finding in the current image study 122 being read.

Once the current image study 122 has been read by compensating for deviations in patient positioning from an optimal patient positioning for a given exam type—e.g., certainty classifications and/or severity grades have been determined—the current image study 122 may, along with the determined certainty classification and/or severity grade, be stored to the database 120. The image studies may be stored to the database 120 by exam type such as, for example, chest x-ray or musculoskeletal imaging and/or by finding (e.g., osteoarthritis, bone fracture). It will be understood by those of skill in the art that newly acquired image studies may be continuously read by compensating for patient positioning deviations, as described above, and stored to the database 120. The neural network 116 may then be trained using the stored exam studies and their corresponding certainty classifications and severity grades.

The processor 102 may be configured to execute computer-executable instructions for operations from applications that provide functionalities to the system 100. For example, the image analysis unit 110 may include instructions for estimating patient positioning parameters, the search engine 112 may include instructions for retrieving previous exam studies based on the patient positioning parameters, the analysis module 114 may include instructions for determining the certainty classification/grading of a suspected finding of the current image study. In another example, the training engine 118 may include instructions for training of the neural network 116. It should be noted, however, that functionalities described with respect to the image analysis unit 110, the search engine 112, the analysis module 114, and the neural network 116 may also be represented as a separately incorporated component of the system 100, a modular component connected to the processor 102 or as a functionalities achievable via more than one processor 102. For example, the system 100 may be comprised of a network of computing systems, each of which includes one or more of the components described above. It will also be understood by those of skill in the art that although the system 100 shows and describes a single neural network 116, the system 100 may include a plurality of neural networks 116, each learning model trained with training data corresponding to a different image study modality, a different target portion of the patient body and/or a different pathology.

In addition, although the exemplary embodiments show and describe the database 120 as being stored to the memory 108, it will be understood by those of skill in the art that the previous exam studies and/or the training data may be acquired from any of a plurality of databases stored by any of a plurality of devices connected to and accessible via the system 100 via, for example, a network connection. In one exemplary embodiment, the previous exam studies may be acquired from one or more remote and/or network memories and stored to a central memory 108. Alternatively, the previous exam studies may be collected and stored to any remote and/or networked memory.

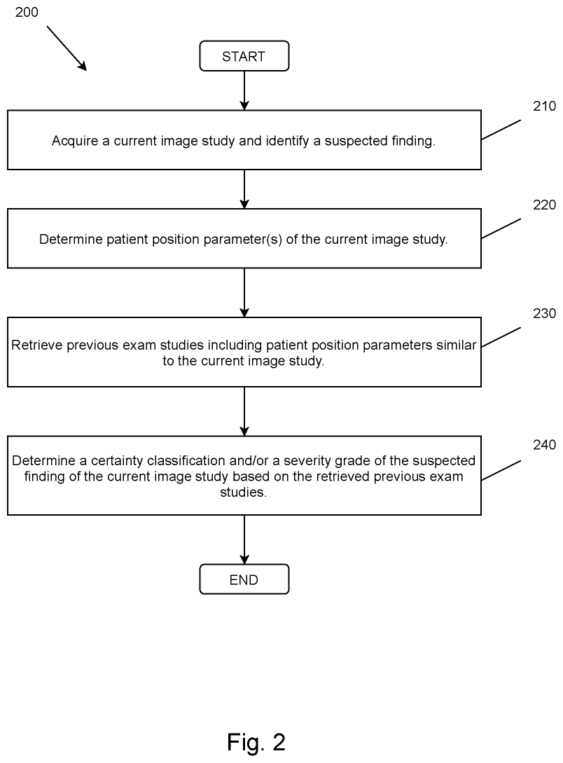

FIG. 2 shows a first exemplary method 200 for compensating for deviations in patient positioning from an optimal position for a current image study 122 by estimating a certainty classification and/or a severity grade based on previous exam studies having similar positioning parameters as the current image study 122. In 210, the current image study 122 is acquired by the imaging device and a suspected finding is identified. In 220, the image analysis unit 110 estimates patient position parameters—i.e., deviations from an optimal positioning based on the image exam type of the current image study 122. As discussed above, if the image exam type is a chest X-ray, these patient position parameters may be measurable values with respect to a rotation along a patient's long axis or an inspiration status of the patient. If the image exam type is a bone X-ray, these patient position parameters may include deviations in geometrical properties of, for example, a joint of the bone.

In 230, the search engine 112 may retrieve previous image studies having the same suspected finding as the current image study 122, from the database 120, and which include similar patient positioning parameters as those identified via the image analysis unit in 220. These previous exam studies may, along with the current image study 122, be displayed to the user on the display 106 so that the images may be compared by the user. Previous image exam studies may include previous diagnostic ratings and may thus be used as a reference when determining a diagnostic rating for the current image study 122. The user may assess the impact of positing on the finding certainty and severity grade based on comparisons with previous cases. In some embodiments, if so desired, additional filters may be applied when retrieving previous image studies. For example, the user may make their own estimation with respect to certainty and/or severity so that the search engine 112 may only retrieve those previous image exams which have a severity grade or certainty classification similar to the user's estimated certainty/severity. In a further embodiment, the previous exam studies may be ranked in priority according to the availability of information confirming the finding of the previous exam study, the severity and/or the certainty. Such additional information may include, for example, follow-up X-rays or images of other modalities (e.g., CT).

Based on the previous image exams that are retrieved via the search engine 112, the user may determine the certainty classification and/or severity grade of the suspected finding of the current image study 122. This user-determined certainty/severity may be stored to the current image study 122 via the analysis module 114. As discussed above, the current image study 122, its findings, certainty and severity, may be stored to the database 120, or a separate database, as training data for the neural network 116.

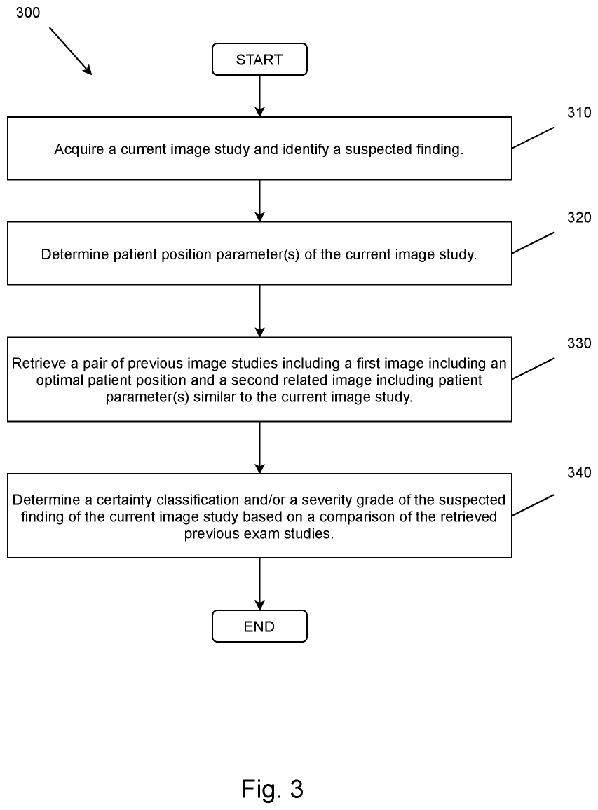

FIG. 3 shows a second exemplary method 300 for compensating for deviations in patient positioning from an optimal position for a current image study 122. The second exemplary method may be substantially similar to the method 200. However, rather than estimating a certainty classification and/or a severity grade based on previous exam studies having similar positioning parameters as the current image study 122, the method 300 estimates a certainty classification and/or severity based on pairs of previously diagnosed images-a first image of an optimal positioning (or close to optimal positioning) and a second image in which the patient positioning deviates from the optimal positioning.

310 and 320 may be substantially similar to 210 220 of the method 200. In particular, in 310, the current image study 122 is acquired by the imaging device and a suspected finding is identified. In 320, the image analysis unit 110 estimates patient position parameters based on the image exam type of the current image study 122.

In 330, however, the search engine 112 retrieves pairs of image exams, preferably of the same patient. In some cases, however, the search engine 112 may retrieve pairs of image exams that are not for the same patient, but which are substantially similar. The first previous image exam includes an image study for the suspected finding, in which the patient is in an optimal position for the image exam type. The second previous image exam incudes an image study for the suspected finding, in which the deviations of the patient positioning parameters are similar to the patient positioning parameters estimated in 320. Thus, in 340, the user may compare the two images to learn the impact of positioning error on a diagnostic rating and may apply the learnings of the comparison toward determining a certainty classification and/or severity grade for the suspected finding of the current image study 122. Similar to the method 200, upon reading of the current image study 122, the current image study 122, along with the user-determined compensation (e.g., determination of certainty classification and/or severity grade), may be stored to the database 120 as a previous image

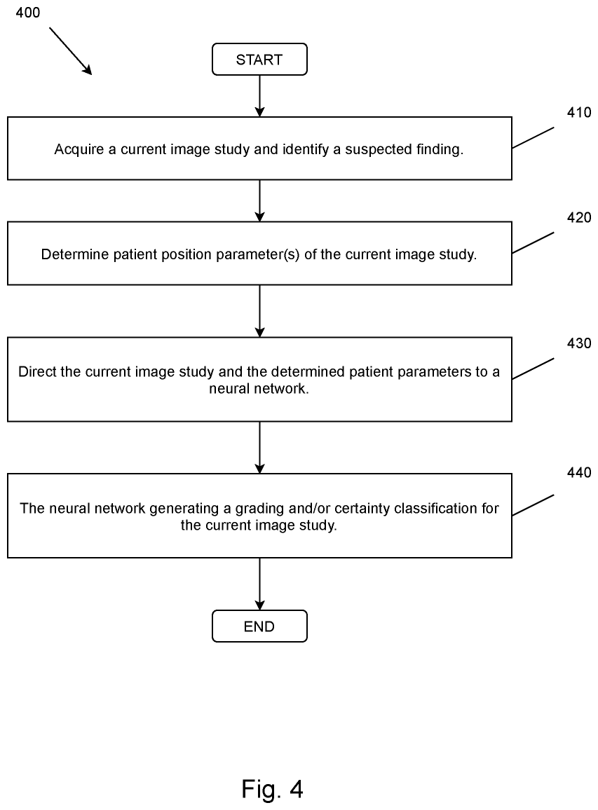

FIG. 4 shows another exemplary method 400 which, similarly to the methods 200, 300 compensates for deviations in patient positioning from an optimal position for a current image study 122. The method 400, however, provides an automatic compensation via the trained neural network 116. Similar to the methods 200, 300, a current image study 122 is acquired by the imaging device and a suspected finding is identified, in 410. In 420, the image analysis unit 110 measures patient position parameters based on the image exam type of the current image study 122.

In 430, however, the current image study 122 and the position parameters measured in 420 are directed to the neural network 116 so that a grading and/or certainty classification for the suspected finding of the current image study 122 is automatically generated. In an exemplary embodiment, the training engine 118 may train the neural network 116 using training data including previous exams from the database 120. Training data may include previous image studies including user-compensations for deviations in patient positioning, as described above with respect to methods 200, 300. Training data may alternatively, or in addition, include previous image studies including deviations in patient positioning from optimal positioning in which a finding, classification and/or severity grade have been confirmed via, for example, additional images.

The neural network 116 may be trained to automatically compensate for the impact of positioning parameters on diagnostic grading based on an image exam type and/or a suspected finding from the image exam. According to an exemplary embodiment, an observed grading (G) of an image including patient position deviations from optimal is automatically decomposed into a real grading (R) that would be observed in an optimal patient position and a deviation in patient positioning (D) relative to the optimal position resulting is measured patient parameters, such that G=R+D. In this embodiment, R is estimated by a forward model, which is trained on the collected training data and maps a given grading G and the measured patient parameters D to the unbiased grading R. In some embodiments, a certainty classification may be similarly automatically generated. Thus, the neural network 116 is trained to automatically generate a predicted grade R for a current image study 122 based on an observed severity grading of the current image study 122 and the measured patient parameters.

Those skilled in the art will understand that the above-described exemplary embodiments may be implemented in any number of manners, including, as a separate software module, as a combination of hardware and software, etc. For example, the image analysis unit 110, the search engine 112, the analysis module 114, the neural network 116 and/or the training engine 118 may be a program including lines of code that, when compiled, may be executed on the processor 102.

Although this application described various embodiments each having different features in various combinations, those skilled in the art will understand that any of the features of one embodiment may be combined with the features of the other embodiments in any manner not specifically disclaimed or which is not functionally or logically inconsistent with the operation of the device or the stated functions of the disclosed embodiments.

It is noted that the claims may include reference signs/numerals in accordance with PCT Rule 6.2(b). However, the present claims should not be considered to be limited to the exemplary embodiments corresponding to the reference signs/numerals.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A computer-implemented method of compensating for image quality issue in an image study, comprising:
measuring, via a processor, a patient positioning parameter indicating a deviation of a patient position in the image study to an optimal patient position for an image exam type of the image study; and
analyzing, via an analysis module, the image study and the determined patient positioning parameter to determine a relationship between positioning errors and a grading of a diagnostic finding for the image study, wherein the grading of the diagnostic finding includes one of a severity of the diagnostic finding or a certainty classification of the diagnostic finding.

2. The method of claim 1, wherein analyzing the image study incudes retrieving, via a search engine, previous image studies from a database, the previous image studies including patient positioning parameters corresponding to the patient positioning parameter determined for the image study.

3. The method of claim 1, wherein analyzing the image study includes retrieving, via a search engine, previous image studies including a first image study including an optimal patient position for diagnostic grading and a second image study including a patient positioning parameter corresponding to the patient positioning parameter of the image study.

4. The method of claim 3, wherein the first and second image studies correspond to the same patient.

5. The method of claim 1, wherein analyzing the image study includes applying a neural network to the image study, the neural network trained using training data including previous image studies including confirmed diagnostic gradings.

6. The method of claim 5, wherein the neural network is trained to generate a predicted grading of a diagnostic finding of the image study.

7. The method of claim 5, wherein the neural network is trained to automatically decompose an observed grading G in a reference component grading R that would be observed in an optimal patient position and offset D is distracted from deviations in patient positioning relative to the optimal patient position.

8. The method of claim 7, wherein G=R+D, R being estimated via a forward model trained using training data mapping a given grading G and the measured patient positioning parameters to an unbiased grading R.

9. The method of claim 1, wherein the image study is an X-ray study.

10. A system compensating for image quality issue in an image study, comprising:
a non-transitory computer readable storage medium storing an executable program; and
a processor executing the executable program to cause the processor to:
measure a patient positioning parameter indicating a deviation of a patient position in the image study to an optimal patient position for an image exam type of the image study; and
analyze the image study and the determined patient positioning parameter to determine a relationship between positioning errors and a grading of a diagnostic finding for the image study, wherein the grading of the diagnostic finding includes one of a severity of the diagnostic finding and a certainty classification of the diagnostic finding.

11. The system of claim 10, wherein the processor executes the executable program to cause the processor to retrieve previous image studies from a database, the previous image studies including patient positioning parameters corresponding to the patient positioning parameter determined for the image study.

12. The system of claim 10, wherein the processor executes the executable program to cause the processor to retrieve previous image studies including a first image study including an optimal patient position for diagnostic grading and a second image study including a patient positioning parameter corresponding to the patient positioning parameter of the image study.

13. The system of claim 12, wherein the processor executes the executable program to cause the processor to retrieve first and second image studies that correspond to the same patient.

14. The system of claim 10, wherein the processor executes the executable program to cause the processor to applying a neural network to the image study, the neural network trained using training data including previous image studies including confirmed diagnostic gradings.

15.-20. (canceled)

21. A non-transitory computer-readable storage medium including a set of instructions executable by a processor, the set of instructions, when executed by the processor, causing the processor to perform operations, comprising:
receiving an image study;
measuring, via a processor, a patient positioning parameter indicating a deviation of a patient position in the image study to an optimal patient position for an image exam type of the image study; and
analyzing, via an analysis module, the image study and the determined patient positioning parameter to determine a relationship between positioning errors and a grading of a diagnostic finding for the image study.

22. The non-transitory computer-readable storage medium of claim 21, wherein analyzing the image study incudes retrieving, via a search engine, previous image studies from a database, the previous image studies including patient positioning parameters corresponding to the patient positioning parameter determined for the image study.

23. The non-transitory computer-readable storage medium of claim 21, wherein analyzing the image study includes retrieving, via a search engine, previous image studies including a first image study including an optimal patient position for diagnostic grading and a second image study including a patient positioning parameter corresponding to the patient positioning parameter of the image study.

24. The non-transitory computer-readable storage medium of claim 23, wherein the first and second image studies correspond to the same patient.

25. The non-transitory computer-readable storage medium of claim 21, wherein analyzing the image study includes applying a neural network to the image study, the neural network trained using training data including previous image studies including confirmed diagnostic gradings.

26. The non-transitory computer-readable storage medium of claim 25, wherein the neural network is trained to generate a predicted grading of a diagnostic finding of the image study.
